(19) United States
(12) Patent Application Publication
(10) Pub. No.: US 2008/0154220 A1
(43) Pub. Date: Jun. 26, 2008

(54) Bag for Use by a Subject Following a Surgical Procedure During Which a Stoma is Formed

(76) Inventor: Charles Gaffney, County Down (GB)

Correspondence Address: DAVID A. GUERRA;INTERNATIONAL PATENT GROUP, LLC, 2025 17TH AVENUE N.W., CALGARY, AB, T2M 0S7, omitted

(21) Appl. No.: 11/815,855

(22) PCT Filed: Feb. 25, 2005
(86) PCT No.: PCT/GB2005/000707

§ 371 Date: Aug. 8, 2007

(30) Foreign Application Priority Data
Feb. 25, 2005 (GB) PCT/GB2005/000707

(51) Int. Cl. A61F 5/441; A61F 5/445; A61F 5/449

(52) U.S. Cl. 604/333; 604/332; 604/339

(57) ABSTRACT

An improved bag for use by a subject following a surgical procedure during which a stoma is formed, the improved bag comprising a partially sealed waterproof pouch having a wall proximal to the body in use and a wall distal from the body in use, the pouch having a lower edge and an upper edge and the proximal wall having an aperture spaced from the upper edge of the pouch to be in registry with a stoma in use, the pouch further comprising a releasable attachment means disposed separate from the aperture for releasably fastening the pouch onto the body of the subject wherein the releasable attachment means is attached to the distal wall of the pouch.

### CROSS-REFERENCE TO RELATED APPLICATIONS

This application is an U.S. national phase application under 35 U.S.C. §371 based upon co-pending International Application No. PCT/GB2005/000707 filed on Feb. 25, 2005. Additionally, this U.S. national phase application claims the benefit of priority of co-pending International Application No. PCT/GB2005/000707 filed on Feb. 25, 2005. The entire disclosures of the prior applications are incorporated herein by reference. The international application was published on Aug. 31, 2006 under Publication No. WO 2006/090093 A1.

### BACKGROUND OF THE INVENTION

1. Field of the Invention

The present invention relates to an improved bag for use by a subject following a colostomy, ileostomy or similar surgical procedure.

2. Description of the Prior Art

Digestive problems and in particular bowel problems result in large numbers of people receiving surgery each year where the outcome of the surgery requires the patient to wear a colostomy bag. The term colostomy bag is used throughout the remainder of the specification but it will be understood to mean any bag suitable for use by a subject following any one of a number of surgical procedures including a colostomy and an ileostomy.

At present, most conventional colostomy bags comprise a flexible waterproof sheet material formed as a pair of walls disposed face to face and permanently secured together along their peripheries to define the bag. One of the bag's walls has an aperture adjacent to but spaced apart from the upper edge of the bag and the aperture is adapted to receive a stoma. Localized attachment means in the form of an adhesive annular stiffening ring is provided around the aperture for attaching the bag to the body wall of the patient.

In the days following a surgical procedure such as a bowel operation, the patient receives bowel irrigation a number of times a day which necessitates frequent bag changes leaving the skin around the stoma irritated, inflamed and very painful. The skin gradually heals when the irrigation treatment is completed and the bag then only needs regular replacement. However, there are still many problems associated with conventional colostomy bags due to the weight of the bag when filling which puts stress on the skin area surrounding the stoma. This results in frequent leakage and generates a high infection risk due to the damaged skin coming into contact with the leaked material. A number of adhesives have been developed in order to obviate or mitigate these problems such as the Eakin™ cohesive and these cohesive materials provide some relief without directly addressing the core problems associated with conventional colostomy bags.

Additionally, when the conventional colostomy bag part fills, the upper portion of the bag is pulled out and down from the body wall of the patient putting further pressure on the skin surrounding the stoma where the bag is attached. As the bag continues to fill, further distortion of the bag causes the wall of the bag distal from the patient's body wall to be pulled down around the aperture of the bag and to block the passage of waste thereby preventing further waste from dropping down into the bag and further exacerbating the problem. In this configuration, the top of the bag projects out from the abdomen and is liable to catch on objects close to the body of the patient. Finally, in this configuration, the contents of the bag are visible through the transparent material which is an undesirable situation for couples wishing to become intimate, for example.

### SUMMARY OF THE INVENTION

It is an object of the present invention to obviate or mitigate the problems of leakage, drooping, waste blockage and waste visibility associated with conventional colostomy bags.

Accordingly, the present invention provides an improved bag for use by a subject following a surgical procedure during which a stoma is formed, the improved bag comprising a partially sealed waterproof pouch having a wall proximal to the body in use and a wall distal from the body in use, the pouch having a lower edge and an upper edge and the proximal wall having an aperture spaced from the edges of the pouch to be in registry with a stoma in use, wherein the pouch further comprises a releasable attachment means disposed separate from the aperture for releasably fastening the pouch onto the body of the subject wherein the releasable attachment means is attached to the distal wall of the pouch.

Ideally, the releasable attachment means is disposed above the aperture in use for releasably fastening the pouch onto the body of the subject above the stoma.

Preferably, the releasable attachment means is disposed adjacent at least a portion of the upper edge of the pouch. Advantageously, the provision of an attachment means adjacent at least a portion of the upper edge of the pouch, above and separate from the aperture in use has a number of technical advantages. The weight of the bag and its contents is now concentrated on the abdomen region where muscle and skin are much stronger than the muscle and tissue around the stoma. As almost none of the weight of the bag is supported around the stoma region, this results in less irritation of the skin in this area due in part to the reduction of friction. Another valuable technical advantage with the location of the attachment means adjacent the upper edge is that the wall of the pouch distal from the body of a person in use is held taut between the lower edge and the upper edge of the pouch. This prevents the previously inherent bunching up of the walls of the pouch around the aperture which is in registry with the waste delivering stoma in use. This is a major technical advance over conventional colostomy bags.

Furthermore, as the weight of the bag and its contents is no longer concentrated around the area of the stoma there is a much reduced occurrence of leaks which reduces the incidence of infection or irritation of skin surrounding the stoma. Finally, the waste material is no longer visible as the top edge of the pouch is attached to the abdomen of the patient. The improved bag has enhanced overall security and safety which increases the confidence levels of the person wearing the device.

Advantageously, a person wearing this bag with an improved aesthetic appearance has greater confidence, security, safety and comfort. In use, the bag is now flatter and more streamlined reducing friction with clothing. Furthermore, when a person wearing the bag is engaged in exercise, there is no longer a need to wear a tight restrictive girdle associated with bags of the prior art, which girdle also had the negative effect of blocking the flow of discharge from the stoma.

Ideally, the releasable attachment means is extending along the upper edge of the pouch and has a flap protruding above the upper edge of the pouch.

Ideally, the flap has a surface proximal to the person's body in use and at least a portion of the proximal surface of the flap has adhesive disposed thereon for adhering the flap to the abdominal region of the person.

Preferably, the entire proximal surface of the flap is covered with the adhesive. Ideally, the releasable attachment means extends over the entire surface of the distal wall of the pouch. Beneficially, the tensile force caused by the weight of the bag and the waste material in the bag is transmitted through the releasable attachment means expanding over the distal wall of the pouch which is held taut and as a result avoiding sagging/clumping of the distal wall around the stoma. As a result, the discharge from the stoma finds its way more freely into the bag.

Advantageously, leaks can now be reduced and ideally controlled to be dry odor leaks. Previously, when a leak occurred, the only weight bearing adhesive was attached to the often delicate, raw skin around the stoma. As a result of the load on the leak cavity, the leak progressed rapidly around the adhesive flange, resulting in an unavoidable total failure of the bag attachment. A dry vertical odor leak can now be easily controlled by retiring discretely for a brief period and effecting a simple repair with a couple of strips of micro pore tape for example, which can be easily kept conveniently at hand. Panic, concern, worry and confusion can be avoided and a person's confidence and peace of mind is improved. The person wearing the bag is no longer a slave to the bag.

Ideally, the adhesive on the flap is protected by a releasable cover prior to use by a patient.

As with conventional colostomy bags, a stiffening annular ring is disposed around the aperture of the improved colostomy bag, the ring having a portion of adhesive and a portion of antiseptic material carried thereon.

Ideally, a reduced volume of adhesive is carried on the annular ring of the improved bag compared to standard bags. This is due to the fact that a substantial volume of adhesive is disposed on the releasable attachment member.

Preferably, the adhesive on the annular stiffening ring provides a waterproof seal between the ring and body preventing the bag and body from separating and generating leaks. The stiffening ring is not now required to carry the weight of the bag and its contents.

Ideally, the colostomy bag has a resealable discharge opening at the lower edge of the pouch. This allows the bag to be emptied intermittently. Preferably, engagement means are disposed along at least one sidewall of the lower edge of the pouch defining the resealable discharge opening. Advantageously, these engagement means allow a subject to easily open the resealable discharge opening for washing the opening or emptying the pouch. The engagement means is a flap on one side wall of the edge or alternatively is a pair of flaps, one on each sidewall.

Ideally, the upper half of the annular stiffening ring in use has a weakened adhesive force compared to the lower half of the annular stiffening ring. As most leaks/bag failures occur as a result of the bag being squeezed in a ballooned state, if the adhesive force acting between the upper half of the annular stiffening ring and a person's body is less than the adhesive force acting between the lower half of the annular stiffening ring and the person's body, then it is more likely the leak will occur on the upper half of the seal between ring and body. This area of the adhesive annular ring is referred to as nine o'clock to three o'clock.

Preferably, there is a reduced volume of adhesive applied on the upper half of the annular stiffening ring.

Alternatively, an adhesive with a reduced adhesive force is applied to the upper half of the annular stiffening ring.

In another embodiment of annular stiffening ring, the surface area of the upper portion of the annular stiffening ring is reduced.

Preferably, the colostomy bag is manufactured in a range of colors.

Ideally, the colostomy bag has designs such as flags, emblems or logos disposed thereon.

Preferably, the colostomy bag may have a covering of lace, frills, or ribbons.

This could help people wearing bags of this type to at least partially overcome confidence issues when meeting partners and/or becoming intimate.

Ideally, the pouch has a gas vent. The gas vent allows body gases to pass out of the pouch to prevent ballooning of the pouch which reduces the number of leaks being generated at the seal between the stoma and annular ring. Ideally, the gas vent is above the level of the stoma. Preferably, a filter is located in fluid communication with the gas vent to deodorize the vented gas.

In a further aspect of the invention there is provided an improved bag for use by a subject following a surgical procedure during which a stoma is formed, the improved bag comprising a partially sealed waterproof pouch having a wall proximal to the body in use and a wall distal from the body in use, the pouch having a lower edge and an upper edge and lateral edges in use and the proximal wall having an aperture spaced from the edges of the pouch to be in registry with a stoma in use, wherein the pouch further comprises a releasable attachment means disposed separate from the aperture wherein the releasable attachment means extends along at least a portion of at least one of the upper edge and lateral edges of the pouch and has a flap protruding beyond the at least one edge of the pouch for releasably fastening the pouch onto the body of the subject.

In a still further aspect of the invention, there is provided an improved bag for use by a subject following a surgical procedure during which a stoma is formed, the improved bag comprising a partially sealed waterproof pouch having a wall proximal to the body in use and a wall distal from the body in use, the pouch having a lower edge and an upper edge and the proximal wall having an aperture spaced from the edges of the pouch to be in registry with a stoma in use, wherein the pouch further comprises a releasable attachment means disposed separate from the aperture wherein the releasable attachment means is disposed on the pouch anywhere other than on the proximal wall for releasably fastening the pouch onto the body of the subject.

### BRIEF DESCRIPTION OF THE DRAWINGS

The invention will now be described with reference to the accompanying drawings, which show by way of example only two embodiments of prior art colostomy bag and one embodiment of improved colostomy bag in accordance with the invention. In the drawings:—

FIG. 1 is a front elevational view of a first embodiment of prior art colostomy bag;

FIG. 2 is a front elevational view of a second embodiment of prior art colostomy bag; and

FIG. 3 is a front elevational view of a first embodiment of an improved colostomy bag.

### DESCRIPTION OF THE PREFERRED EMBODIMENT

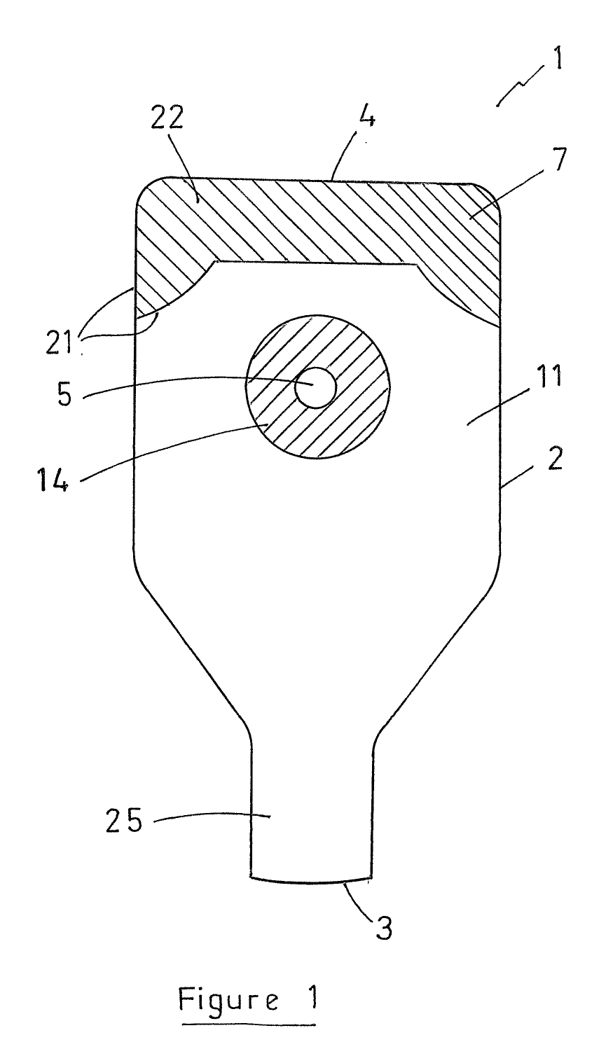

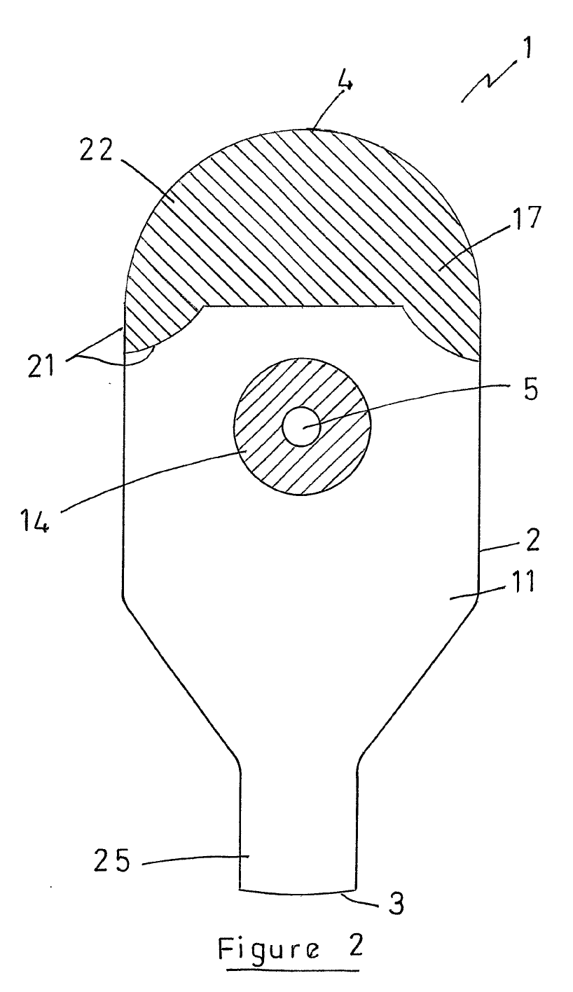

In FIG. 1 and FIG. 2 of the drawings there is shown a colostomy bag indicated generally by the reference numeral 1. The colostomy bag 1 has a waterproof partially sealed pouch 2 having a wall 11 proximal to the body in use and a wall (not shown) distal from the body in use. The pouch has a lower edge 3 and an upper edge 4 when mounted on a person's body in use. Proximal wall 11 of the bag 1 has an aperture 5 defined therein, the aperture 5 being spaced apart from the upper edge 4. The aperture 5 is in register with a stoma (not shown) in use. An annular ring 14 is disposed on the proximal wall 11 around the aperture 5 and the ring 14 has a portion of adhesive and a portion of antiseptic material disposed thereon. There is a full ring of adhesive to provide the waterproof seal.

Attachment members 7, 17 are disposed on the proximal walls 11 of the bags 1 of FIG. 1 and FIG. 2 respectively and extend along the upper edge 4 of the respective pouches 2. It will of course be appreciated that the shape of the attachment members 7, 17 is not limited to the two embodiments shown as it will be apparent to one of ordinary skill in the art that many shapes and sizes of attachment member will provide the same function and the attachment member may include such arrangements as a plurality of strips or patches.

The attachment members 7, 17 comprise a strip 21 of flexible material having one surface attached to the proximal wall 11 and one exposed surface 22 having an adhesive carried thereon. The upper edge 4 of the pouch 2 of FIG. 1 is flat whereas the upper edge 4 of the pouch 2 of FIG. 2 is arcuate and the shape of the upper edge of the strips of both attachment members 7, 17 correspond to the shape of the upper edges 4 of the respective pouches 2. The strips 21 and the annular rings 14 can have releasable pull-off masking (not shown) located thereon prior to applying the bag 1 to the body in order to protect the adhesive. The bags 1 have a resealable discharge opening 25 located along their lower edge 3.

In use, a person peels the cover off the annular ring 14 and applies the annular ring 14 onto the skin around the stoma. The person then removes the cover from the strip 21 and adheres the exposed surface 22 of the strip to their abdomen region. The adhesive on the strip takes the weight of the bag and its contents which means the adhesive on the annular ring 14 has little or no load bearing requirement and is only required to seal the ring 14 to the skin around the stoma. However, a significant portion of the proximal wall 11 is covered with adhesive and the portion of the wall 11 carrying the adhesive is unable to move relative to the body when a person adjusts their bodily position.

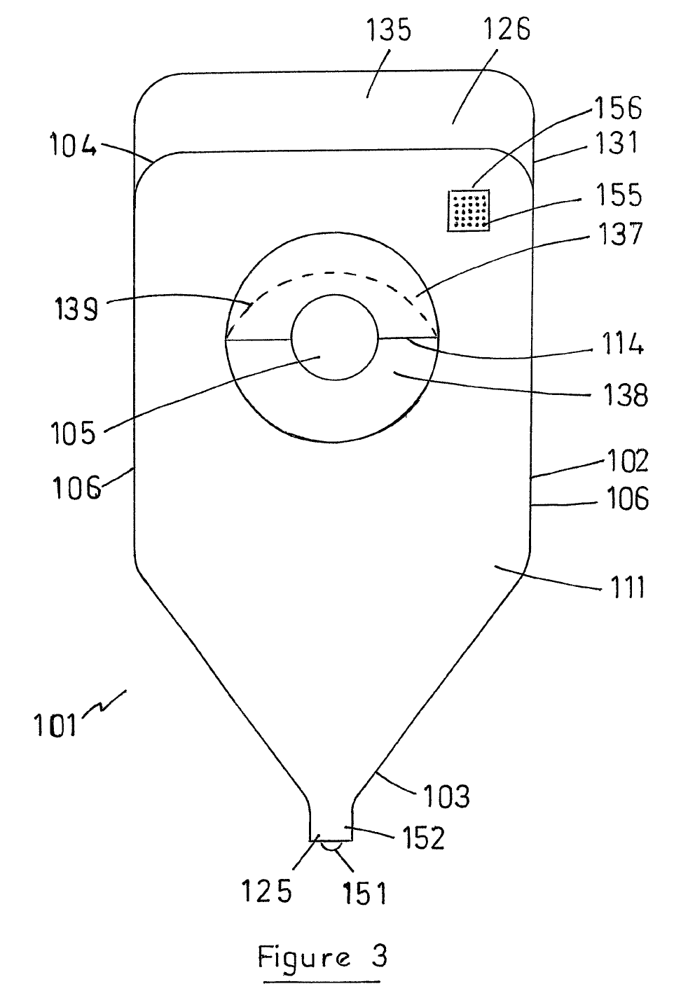

In FIG. 3 of the drawings there is shown an improved colostomy bag indicated generally by the reference numeral 101 in accordance with the invention. The colostomy bag 101 has a waterproof partially sealed pouch 102 having a wall 111 proximal to the body in use and a wall (not shown) distal from the body in use. The pouch 102 has a lower edge 103 and an upper edge 104 and two lateral edges 106 when mounted on a person's body in use. Proximal wall 111 of the bag 101 has an aperture 105 defined therein, the aperture 105 being spaced apart from the edges 103, 104 and 106. The aperture 105 is in register with a stoma (not shown) in use. An annular ring 114 is disposed on the proximal wall 111 around the aperture 105 and the ring 114 has a portion of adhesive and a portion of antiseptic material disposed thereon. There is a full ring of adhesive to provide the waterproof seal.

A releasable attachment member 131 is attached to the wall of the bag 101 distal from a person's body in use. The releasable attachment member 131 may be adhered to a portion of the distal wall adjacent at least a portion of the upper edge 104. Alternatively, the releasable attachment member 131 is adhered over the entire surface of the distal wall of the bag 101. The releasable attachment member 131 has a flap 135 protruding beyond the upper edge 104 of the bag 101. The surface 126 of the flap 135 proximal to a person's body in use carries an adhesive for adhering the flap 135 to the person's body above the stoma. The effect of this flap 135 is to allow the entire surface of the proximal wall 111 to be free from adhesive so that the proximal wall 111 has greater flexibility to move relative to the body of the subject as the subject sits down or gets up from the sitting position, ascends or descends stairs or enters or leaves a vehicle for example.

The annular stiffening ring 114 is divided into an upper half 137 and a lower half 138. The adhesive force acting between the upper half 137 of the ring 114 and the person's body is less than the adhesive force acting between the lower half 138 of the ring 114 and the person's body to encourage unavoidable leaks to occur between the upper half 137 of the ring 114 and the person's body. It will of course be appreciated that the weakened adhesive force may be applied to a more limited range of the annular stiffening ring 114 than the area from nine o'clock around to three o'clock. This feature of the invention would work equally as well with the weakened adhesive force acting between the surface area of the ring 114 defined from ten o'clock around to two o'clock. Broken line 139 is provided on the upper half 137 of the annular stiffening ring 114 to show a person the line to cut along to reduce the surface area of the upper half 137 of the ring 114 in order to reduce the adhesive force between the upper half 137 and the person's body particularly if the adhesive force of the adhesive on the upper and lower halves is the same. Resealable discharge opening 125 has a flap 151 located along one of the side walls 152 of the lower edge 103 defining the opening 125. The flap 151 can be gripped by a subject between their index finger and thumb to increase the ease with which the resealable discharge opening 125 can be opened. It will be appreciated that any number of flaps 151 can be used anywhere proximal to the opening 125 to achieve the result of the subject more easily separating the side walls 152 which define the opening 125. A gas vent 155 is located on the distal wall of the pouch 102 to allow body gas to pass out of the pouch 102. A filter 156 encloses the gas vent 155 to deodorize the body gas before it enters the atmosphere.

Variations and modifications can be made without departing from the scope of the invention defined in the appended claims.

1-15. (canceled)

16. A colostomy bag system for use by a subject following a surgical procedure during which a stoma is formed, said bag comprising:
a partially sealed waterproof pouch having a wall proximal to the body in use and a wall distal from a subjects body in use, said pouch having a lower edge and an upper edge, said proximal wall defining an aperture spaced from said edges of said pouch to be in registry with a stoma in use; and
a releasable attachment means disposed separate from said aperture on said pouch for releasably fastening said pouch onto the body of the subject, wherein said releasable attachment means is attached to said distal wall of said pouch.

17. The colostomy bag system as claimed in claim 16, wherein said releasable attachment means is disposed adjacent at least a portion of said upper edge of said pouch.

18. The colostomy bag system as claimed in claim 17, wherein said releasable attachment means extends along said upper edge of said pouch and has a flap protruding above said upper edge of said pouch.

19. The colostomy bag system as claimed in claim 18, wherein said flap has a surface proximal to the subject's body in use and at least a portion of said proximal surface of said flap has adhesive disposed thereon for adhering said flap to the abdominal region of the person.

20. The colostomy bag system as claimed in claim 19, wherein said entire proximal surface of said flap is covered with adhesive.

21. The colostomy bag system as claimed in claim 16, wherein said releasable attachment means extends over the entire surface of said distal wall of said pouch.

22. The colostomy bag system as claimed in claim 16 further comprising an annular stiffening ring disposed around said aperture of said pouch, said annular stiffening ring having a portion of adhesive carried thereon.

23. The colostomy bag system as claimed in claim 22, wherein an upper portion of said annular stiffening ring in use has a weakened adhesive force compared to the adhesive force of a lower portion of said annular stiffening ring.

24. The colostomy bag system as claimed in claim 23, wherein said annular stiffening ring further comprising a broken line provided on said upper portion of said annular stiffening ring, said broken line provides a cutting guide so as to reduce the surface area of said upper portion of said stiffening ring in order to reduce the adhesive force between said upper portion and the body in use.

25. The colostomy bag system as claimed in claim 16 further comprising a covering made of material selected from the group consisting of lace, frills and ribbons.

26. The colostomy bag system as claimed in claim 16 further comprising a gas vent attached to said pouch.

27. The colostomy bag system as claimed in claim 26 further comprising a filter located in fluid communication with said gas vent.

28. The colostomy bag system as claimed in claim 16, wherein said lower edge of said pouch has a pair of sidewalls defining a resealable opening therebetween, and a flap disposed along at least one of said sidewalls of said lower edge of said pouch.

29. An improved colostomy bag comprising:
a partially sealed waterproof pouch having a wall proximal to the body in use and a wall distal from a subjects body in use, said pouch having a lower edge, an upper edge, and lateral edges, said proximal wall defining an aperture spaced from said edges of said pouch to be in registry with a stoma in use; and
a releasable attachment means disposed separate from said aperture and extends along at least a portion of at least one of said upper and later edges of said pouch, said releasable attachment means further comprising a flap protruding beyond said at least one edge of said pouch for releasably fastening said pouch onto the body of the subject.

30. The colostomy bag system as claimed in claim 29 further comprising an annular stiffening ring disposed around said aperture of said pouch, said stiffening ring having a portion of adhesive carried thereon.

31. The colostomy bag system as claimed in claim 30, wherein an upper portion of said annular stiffening ring in use has a weakened adhesive force compared to the adhesive force of a lower portion of said annular stiffening ring.

32. The colostomy bag system as claimed in claim 31, wherein said annular stiffening ring further comprising a broken line provided on said upper portion of said annular stiffening ring, said broken line provides a cutting guide so as to reduce the surface area of said upper portion of said stiffening ring in order to reduce the adhesive force between said upper portion and the body in use.

33. The colostomy bag system as claimed in claim 30 further comprising a gas vent attached to said pouch, and a filter located in fluid communication with said gas vent.

34. The colostomy bag system as claimed in claim 30, wherein said lower edge of said pouch has a pair of sidewalls defining a resealable opening therebetween, and a flap disposed along at least one of said sidewalls of said lower edge of said pouch.

35. An improved colostomy bag comprising:
a partially sealed waterproof pouch having a wall proximal to the body in use and a wall distal from a subjects body in use, said pouch having a lower edge, an upper edge, and lateral edges, said proximal wall defining an aperture spaced from said edges of said pouch to be in registry with a stoma in use;
a flap protruding above said upper edge of said pouch, said flap having a surface proximal to the subject's body in use and at least a portion of said proximal surface of said flap has adhesive disposed thereon for adhering said flap to the abdominal region of the person;
an annular stiffening ring disposed around said aperture of said pouch, said stiffening ring having a portion of adhesive carried thereon, wherein an upper portion of said annular stiffening ring in use has a weakened adhesive force compared to the adhesive force of a lower portion of said annular stiffening ring, and wherein said annular stiffening ring further comprising a broken line provided on said upper portion of said annular stiffening ring, said broken line providing a cutting guide so as to reduce the surface area of said upper portion of said stiffening ring in order to reduce the adhesive force between said upper portion and the body in use;
a gas vent located adjacent said upper edge of said pouch; and
a filter located in fluid communication with said gas vent.
